(19) United States
(12) Patent Application Publication
(10) Pub. No.: US 2015/0080943 A1
(43) Pub. Date: Mar. 19, 2015

(54) Tourniquet

(71) Applicant: SILICAMED, LTD, HONG KONG (CN)

(72) Inventor: Hui Juan YE, Hong Kong (CN)

(73) Assignee: SILICAMED, LTD, HONG KONG (CN)

(21) Appl. No.: 14/340,628

(22) Filed: Jul. 25, 2014

(30) Foreign Application Priority Data
Sep. 19, 2013 (GB) 1316616.0

(51) Int. Cl. A61B 17/132

(52) U.S. Cl. CPC A61B 17/132
USPC 606/203

(57) ABSTRACT

A disposable tourniquet comprising a buckle and a band attached at one end to the buckle, the buckle comprising a base into which the band can be laid and a laterally hinged clamp closure that can be closed over the band to grip it in the buckle and a hinged closure securing tab.

### FIELD OF THE INVENTION

This invention relates to tourniquets, primarily for haemostasis.

### BACKGROUND OF THE INVENTION

Tourniquets are essentially bands that wrap tightly around a limb to restrict blood supply, and, in its simplest form, a tourniquet is simply a band with a means for holding it tightly on the limb, and that means can be as simple as a knot.

However, for better control of tension and ease of use, the means often comprise a buckle. Tourniquets are known with sophisticated buckle and tensioning arrangements, intended for single-handed use. However, such arrangements are not suitable for disposable tourniquets, for which there is considerable demand. Simple, inexpensive buckle arrangements have been proposed for disposable tourniquets, which, however, have various drawbacks.

A simple pronged buckle is known, which works in an obvious and familiar fashion, but which has limited tension flexibility, as the available tensions are dictated by the spacing of holes for the prong, and the belt needs to be overtensioned in order to locate the prong in the selected hole, and to release it, which can be uncomfortable, if not, indeed, harmful to the patient.

Hook and loop fastening arrangements are also known, hut they need manipulation involving pressure and tension to secure and release them, this, again, may impact on the patient. They have the added disadvantage that the hook component can lock on to fabrics, such as bandages, it comes into contact with, which can cause unwanted complications in an emergency procedure.

Other known arrangements are not very secure, and not easy to release in controlled manner.

### BRIEF DESCRIPTION OF THE INVENTION

The present invention comprises a disposable tourniquet comprising a buckle and a band attached at one end to the buckle, the buckle comprising a base into which the band can be laid and a laterally hinged clamp closure that can be closed over the band to grip it in the buckle and a hinged closure securing tab.

The buckle may be moulded in plastics material and the closure hinge can be a live hinge, as, indeed, be the tab hinge.

This arrangement enables the tourniquet to be correctly tensioned without the need to overtension and the clamp closure to he closed, gripping the band, with a simple finger and thumb action, and to be opened in controlled fashion, as the lid can be held down on to the band by a finger while the thumb is releasing the tab, And this action can also gradually release lid pressure on the band, allowing a gradual release of band tension.

The tourniquet may have a tension confirming arrangement, indicating when a correct tension is being applied. The band may be elastic, and have a marking comprising a compressed geometrical shape that stretches to ‘look’ right at the correct tension. Such a shape may comprise a parallelogram that stretches to become a square, Such shapes may be printed all along the band.

The buckle base and/or closure may comprise a gripping formation such as ribbing, the better to grip the band.

### BRIEF DESCRIPTION OF THE DRAWINGS

One embodiment of tourniquet according to the invention will now be describer with reference to the accompanying drawings, in which:

FIG. 1 is a front view of the tourniquet;

FIG. 2 is a view on Arrow II of FIG. 1;

FIG. 3 is a perspective view of one embodiment of buckle of the tourniquet of FIGS. 1 and 2:

FIG. 4A is a first view of a tension confirming arrangement for the tourniquet of FIGS. 1 to 3;

FIG. 4B is a second view of a tension confirming arrangement for the tourniquet of FIGS. 1 to 3;

FIG. 5 is a side elevation of another embodiment of buckle, open;

FIG. 6 is an elevation of the buckle of FIG. 5, closed; and

FIG. 7 is a view from the top o the buckle of FIG. 5.

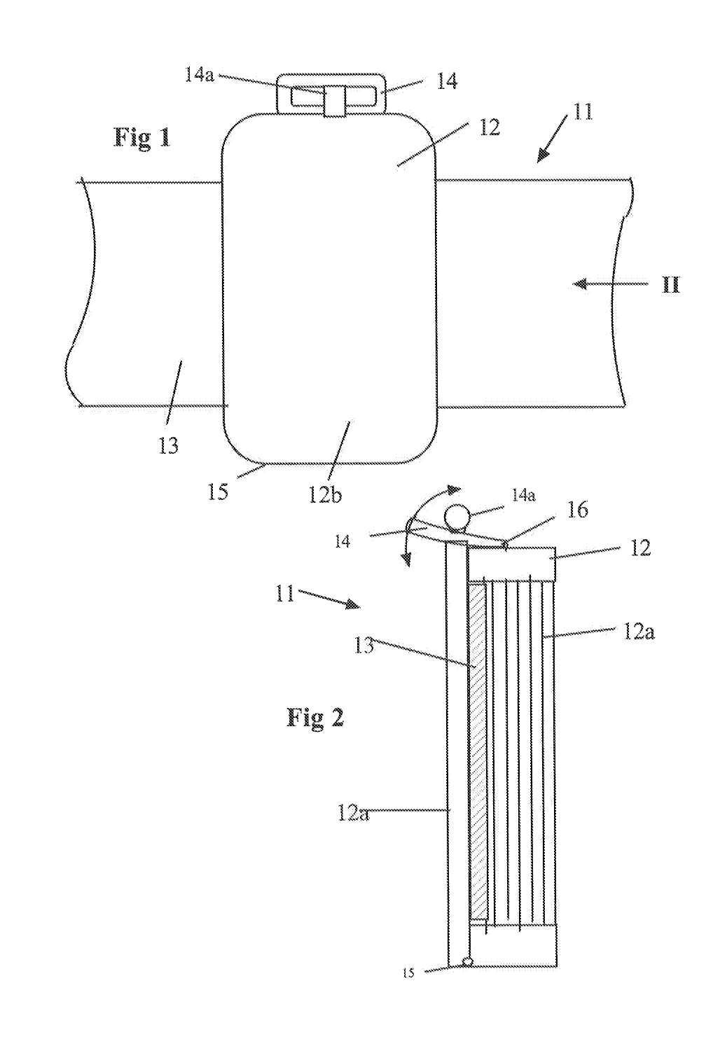

### DETAILED DESCRIPTION OF THE INVENTION

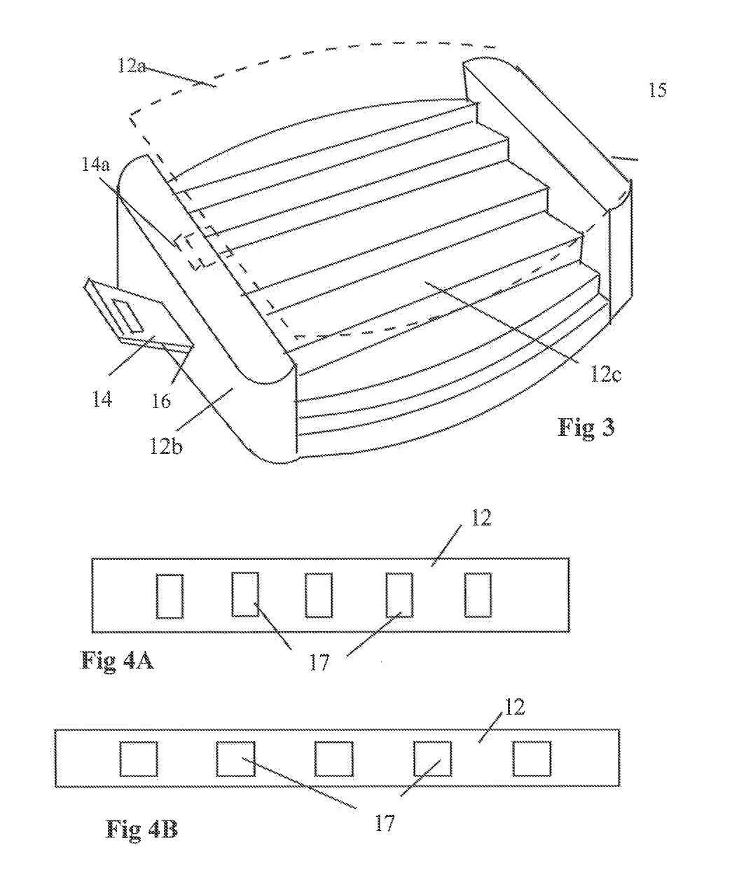

The drawings illustrate a disposable tourniquet 11 comprising a buckle 12 and a band 13 attached at one end to the buckle 12, the buckle 12 comprising a base 12a into which the band 13 can be laid and a laterally hinged clamp closure 12b (shown in broken line in FIG. 3) that can be closed over the band 13 to grip it in the buckle 12, and a hinged closure securing tab 14 that clips over a projection 14a of the closure 12b.

The buckle 12 is moulded in plastics material and the closure hinge 15 is a live hinge, as, indeed, is the tab hinge 16,

This arrangement enables the tourniquet 11 to be correctly tensioned without the need to overtension and the clamp closure 12b to be closed, gripping the band 13, with a simple finger and thumb action, and to be opened in controlled fashion, as the closure 12b can be held down on to the band 13 by a finger while the thumb is releasing the tab 14. And this action can also gradually release closure pressure on the band 13, allowing a gradual release of band tension.

The tourniquet 11 has a tension confirming arrangement, indicating when a correct tension is being applied. The band 13 is elastic, and has a marking comprising a number of compressed geometrical shapes 17 that stretch to ‘look’ right at the correct tension. The shape as illustrated comprises a rectangle that stretches to become a square. Such shapes are printed all along the band, FIG. 4a shows the band 13 relaxed, FIG. 4a shows it stretched to the correct tension.

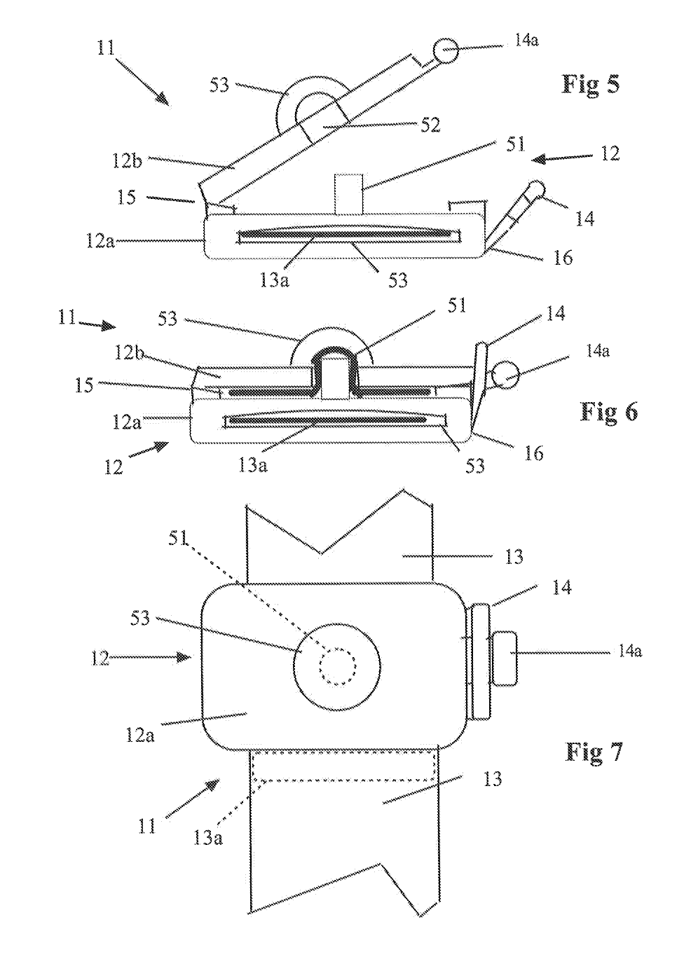

The buckle base 12a comprises a gripping formation of ribbing 12c, the better to grip the band, Another gripping arrangement is shown in FIGS. 5 and 6, where a post 51 is upstanding from the base 12a which bends the band 13 into a complementary aperture 52 concealed by a cap 53 in the closure 12b.

The base 12a has a slot 53 in which captive end 13a of the band 13 is held—see particularly FIG. 7—by terminating in a thickened rib that cannot be pulled through.

What is claimed:

1. A disposable tourniquet comprising a buckle and a band attached at one end to the buckle, the buckle comprising a base into which the band can be laid and a laterally hinged clamp closure that can be closed over the band to grip it in the buckle and a hinged closure securing tab.

2. A disposable tourniquet according to claim 1, in which the buckle is moulded in plastics material and the closure hinge is a live hinge

3. A disposable tourniquet according to claim 2, in which the tab hinge is a live hinge.

4. A disposable tourniquet according to claim 1, having a tension confirming arrangement, indicating when a correct tension is being applied.

5. A disposable tourniquet according to claim 4, in which the band is elastic, and have a marking comprising a compressed geometrical shape that stretches to ‘look’ right at the correct tension.

6. A disposable tourniquet according to claim 5, in which the shape comprises a parallelogram that stretches to become a square.

7. A disposable tourniquet according to claim 6, in which such shapes are printed all along the band.

8. A disposable tourniquet according to claim 1, in which the buckle base and/or closure comprises a gripping formation.

9. A disposable tourniquet according to claim 8, in which the gripping formation comprises ribbing.

10. A disposable tourniquet according to claim 8, in which the gripping formation comprises a post upstanding from one of the base and the closure and a complementary aperture in the other into which the band is jammed by the post.
